(19) United States
(12) Patent Application Publication
(10) Pub. No.: US 2018/0080929 A1
(43) Pub. Date: Mar. 22, 2018

(54) ANALYTICAL DEVICE

(71) Applicants: National University Corporation Nagoya University, Aichi (JP); National University Corporation Hokkaido University, Hokkaido (JP)

(72) Inventors: Toshihiro KASAMA, Aichi (JP); Yoshinobu BABA, Aichi (JP); Manabu TOKESHI, Hokkaido (JP); Nanako NISHIWAKI, Hokkaido (JP)

(21) Appl. No.: 15/560,921

(22) PCT Filed: Mar. 17, 2016
(86) PCT No.: PCT/JP2016/058447

§ 371 Date: Sep. 22, 2017

(30) Foreign Application Priority Data
Mar. 24, 2015 (JP) 2015-060845

(51) Int. Cl. G01N 33/543; B01L 3/00

(52) U.S. Cl. CPC G01N 33/54366; B01L 3/502761; B01L 3/502707; B01L 2200/12; B01L 2300/0858; B01L 2300/161; B01L 2300/0654; G01N 33/54306

(57) ABSTRACT

The present invention provides a device capable of detecting a plurality of items in a specimen using a single optical system. Microstructures holding specific binding reagents in a photocured hydrophilic resin are arranged in a channel, whereby the reagents do not mix during manufacture of the device, and a device capable of multiplex detection can be manufactured.

### BACKGROUND OF THE INVENTION

### 1. Field of the Invention

The present invention relates to: a testing device that uses specific binding; a testing kit; and a testing system. The present invention also relates to a method of manufacturing the device. In particular, the present invention relates to a microfluidic device provided with a microchannel and capable of analyzing a plurality of items at once.

### 2. Description of the Related Art

Analysis methods that use specific binding, as in an immunoassay utilizing the affinity of an antibody for an antigen, have been widely used conventionally in clinical testing and the drug discovery field. Because a small amount of a specimen and/or reagent to be used suffices, and because the amount of time required for an assay can be reduced, assays using a microdevice having a small reaction system capacity have been developed in place of assays using a plate such as the so-called 96-well plate.

Moreover, as compared to using a plate as a carrier, by using channels, the amount of time required for an assay is reduced. The inventors of the present invention have also disclosed microfluidic devices for immunoassay in which beads having an antibody in the solid phase are arranged in a channel, as a pillar-like structure where the beads are homogeneously dispersed and held in a photocured hydrophilic resin (Patent reference 1, Non-patent reference 1).

FIG. 8A illustrates a microfluidic device where a primary antibody is immobilized on polystyrene microbeads having a diameter of approximately 1 μm, and a photocurable resin provides a pillar-like microstructure within channels. The microfluidic device is a single-item analysis device in which the microbeads having the primary antibody are suspended in a hydrophilic photocurable resin solution, are filled into the channels while in the suspension, and are patterned and cured by an exposure process.

This device is used as follows. First, a specimen such as blood serum or urine is filled into the channels, is incubated, and an antigen contained in the specimen is bound to the primary antibody carried by the beads. After the specimen is washed with a washing solution, a fluorescent-labeled antibody is injected and incubated, and is bound to the antigen (substance to be detected) that is bound to the primary antibody. Next, the fluorescent-labeled antibody that has not bounded is washed with the washing solution, after which fluorescence of the labeled antibody is detected by a fluorescent detector.

FIG. 8B illustrates a triplex analysis device capable of detecting three different antigens in one assay. This device mixes together three types of microbeads each having antibody that recognizes the different antigens and suspends the microbeads in a photocurable resin, then performs a similar exposure process to cure the suspension in the channels, whereby the suspension is fixed.

In this method, the three types of microbeads having the different antibodies are fixed inside a single pillar-like structure. These are detected using secondary antibodies that are conjugated with three different types of fluorescent dyes. At this point, the three types of antigens and the fluorescent labels can form bonds inside the same structure, and therefore fluorescent dyes that are excited by different wavelengths must be used as the fluorescent dyes used for detection. Also, fluorescent labels must be used that have excitation spectra and fluorescent spectra that are sufficiently far apart so that the signal originating from each fluorescent label is not observed as mixed. For example, fluorescent dyes such as FITC, Alexa FLUOR 555 (trademark), or DyLight 650 (trademark) must be used as the fluorescent labels for the antibodies used for detection.

[Patent Document 1] Japanese Patent No. 4,717,081

[Patent Document 2] Japanese Examined Patent Application No. S55-40

[Patent Document 3] Japanese Examined Patent Application No. S55-20676

[Patent Document 4] Japanese Examined Patent Application No. S62-19837

[Patent Document 5] Japanese Laid-open Patent Application No. 2009-48833

[Non-patent Document 1] Ikami M., et al., Lab on a Chip, 2010, Vol. 10, pp. 3335-3340

[Non-patent Document 2] Ito, Y., et al., Biomaterials, 2005, Vol. 26, pp. 211-216

[Non-patent Document 3] Ito, Y., & Nagawa, M., Biomaterials, 2003, Vol. 24, pp. 3021-3026

### SUMMARY OF THE INVENTION

Because it suffices to use a single fluorescence-detecting detector for the single-item analysis device shown in FIG. 8A, the detector is also compact, and detection can be performed in a short amount of time with favorable sensitivity. Despite this, there has been the problem that the device is not capable of multiplex analysis. Also, in the case of the triplex analysis device shown in FIG. 8B, a light source having sufficient separation between the excitation spectra must be selected, and therefore the most that can be detected by a single device is three types of antigens. In addition, optical systems matched to the types of fluorescent dye are required, and an apparatus switching between the optical systems in order to switch between a plurality of optical filters is also required. Therefore, reducing the size and price of a fluorescent detector is difficult.

Given this, in order to detect a plurality of items with a single device, an attempt was made to severally fix the different beads to the channel, by preparing a photocurable resin having beads with different antibodies and performed multiple exposures (FIG. 9).

As shown in FIG. 9A, beads having a single primary antibody A in the solid phase were suspended in a photocurable resin solution, and the suspension was filled into the channel. The channel was covered by a photomask provided with an opening such that exposure was possible only at a portion where curing of the resin was desired, and the resin was cured by UV irradiation. The uncured resin was washed and evacuated from the channel, whereby the A antibody immobilized on beads were fixed to the channel (FIG. 9A, right side). Next, beads immobilized a B antibody were suspended in a photocurable resin solution, and the suspension was filled into the channel (FIG. 9B). The channel was covered by a photomask configured such that exposure was possible only at a portion where curing of the resin was desired, and the B antibody immobilized on beads were cured by UV irradiation together with the photocurable resin. After the resin was photocured, the uncured resin was washed.

With this method, a structure was created in which beads having different antibodies in the solid phase were sequentially cured, together with the photocurable resin, in different portions of the channel (FIG. 9C). FIG. 9D illustrates results of a measurement made using the multiplex device manufactured with this method, using the beads coated with antibodies.

The multiplex device of FIG. 9D was manufactured as follows. An anti-CRP antibody and anti-CEA antibody were used, each respectively coated onto polystyrene beads. A mixed resin solution in which a photo-cross-linkable polymer solution having polyethylene glycol as a basic skeleton (MI-1, manufactured by Kansai Paint Co., Ltd.) and a photocuring initiator solution (PIR-1, manufactured by Kansai Paint Co., Ltd.) were mixed with purified water was used as the photocurable resin.

A mixed solution of the mixed resin solution and the beads coated with the anti-CEA antibody was filled into the channel, was irradiated with ultraviolet rays to photocure a mixed product of the resin and antibody-coated beads, and a wall-like parallelepiped structure was produced inside the channel. The uncured resin was suctioned away and washing was performed with a washing solution (corresponds to A antibody immobilized bead fixing step, FIG. 9A).

Next, the mixed resin solution and the anti-CRP antibody-coated beads were mixed, and the mixture was fixed to a different region from the region where the anti-CEA antibody is fixed (corresponds to B antibody immobilized on bead fixing step, FIG. 9B). A device for assaying a combination of items was manufactured having the anti-CEA antibody and the anti-CRP antibody fixed in the same channel.

However, when detection was performed using as the specimen a solution containing only the CRP antigen, fluorescence was also detected within the region where the anti-CEA antibody was fixed (upper right of FIG. 9D, CEA fluorescent micrograph). As depicted schematically in FIG. 9C, this is believed to be the anti-CRP antibody-coated beads caught on the first photocured region. When observed with a scanning electron microscope, the cured photocurable resin had a porous structure. Therefore, it is believed that in the process of manufacturing the device, the beads are caught in fine holes in the cured resin, and a plurality of types of antibody immobilized on beads are contained in a single pillar-like structure because the beads are embedded in the structure. In other words, in the process of washing the uncured resin, the antibody immobilized on beads are believed to become embedded in fine holes in the structure.

In order to resolve the problems noted above, contamination that may occur when a plurality of antibodies enter the solid phase can be prevented by using a microarray, rather than a microchannel system. Microarrays using a photocurable resin have been disclosed previously, but in all cases the resin has been applied, and a protein (antibody, etc.) solution has been applied over the resin, after which photocuring is performed (Non-patent Documents 2 and 3). Therefore, a reaction site where a specific binding reagent such as an antibody reacts with the substance to be detected is limited exclusively to the surface of the photocurable resin onto which the specific binding reagent is applied. As a result, there has been the problem that the reaction site is constrained and detection sensitivity is low. In addition, the specific binding reagent enters the solid phase due to being applied to the surface, leading to differences in the amount of the immobilized specific binding reagent between lots. Therefore, there has been a problem of significant discrepancies between lots.

The present invention has been contrived in order to resolve the above-noted problems, and provides a microfluidic device that is capable of detecting even a plurality of items at once with a single optical system, while preserving the strengths of a device having a microchannel, which achieves detection results in a short amount of time with favorable sensitivity. Specifically, the present invention provides a method for severally fixing specific binding reagents to a plurality of structures in a channel.

### Means for Solving the Problems

The present invention relates to the below-noted microfluidic device, analysis kit, analysis system for microfluidic device, and manufacturing method of microfluidic device.

(1) A microfluidic device having at least one channel on a substrate, at least one microstructure being arranged within each of the channels, and, in the microstructures, one type of specific binding reagent or one type of specimen into a photocured hydrophilic resin being held directly in the hydrophilic resin through cross-linking.

(2) The microfluidic device of (1), wherein, in each of the microstructures, a different specific binding reagent and/or specimen is directly held in the hydrophilic resin through cross-linking.

(3) The microfluidic device of (1) or (2), wherein the specific binding reagent is at least one of an antibody, antigen, avidin, streptavidin, or biotin.

(4) The microfluidic device of any one of (1) to (3), wherein the specimen comprises a cell, cell cluster, cell membrane, organelle, or exosome.

(5) An analysis kit, comprising:

the microfluidic device of any one of (1) to (4); and

a labeled reagent that binds specifically to a substance to be detected.

(6) The analysis kit of (5), wherein the labeled reagent can be detected by a single optical system.

(7) A system using the microfluidic device of any one of (1) to (4), a measurement start means for performing measurement, a detection means for detecting fluorescence intensity by a single fluorescence while scanning over the microfluidic device, and a display means for displaying fluorescence intensity as a numerical value.

(8) A method of manufacturing a microfluidic device, comprising:

a substrate preparation step of preparing a substrate comprising at least one channel; a filling step of filling an interior of the channel with a mixed solution of one type of specific binding reagent or one type of specimen and a hydrophilic photocurable resin;

an exposure step of performing exposure to light using a photomask in which a structure is formed in a portion of the hydrophilic photocurable resin filled into the channel, and of cross-linking and photocuring the specific binding reagent or specimen directly with a resin;

a washing step of removing and washing uncured resin from the channel.

(9) A method of manufacturing a microfluidic device of (8), after the washing step, wherein a refilling step of filling the interior of the channel with a mixed solution of a specific binding reagent or specimen different from the said specific binding reagent or specimen and a hydrophilic photocurable resin; a re-exposure step of exposing the hydrophilic photocurable resin filled into the channel to light using a photomask in which a structure is formed on a site where uncured photocurable resin is present; a re-washing step of removing and washing uncured resin from the channel; and repeating the steps from the refilling step to the re-washing step and fixing a plurality of different specific binding reagents and/or specimens inside the channel.

### Effects of the Invention

Up until now, there has been no device where a specific binding reagent and a photocurable resin were directly mixed and entered the solid phase through cross-linking. According to the method of the present invention, a specific binding reagent can be fixed to a channel without using microbeads, and therefore there is no commingling of the specific binding reagents fixed to various structures. A different reagent can be fixed to each microstructure, and therefore different substances to be detected can be detected depending on a detection reagent assigned a single label. As a result, a detector can be made smaller, and a plurality of items can be analyzed with a simpler system.

### BRIEF DESCRIPTION OF THE DRAWINGS

FIG. 1 illustrates a procedure for a method of manufacturing a microfluidic device;

FIGS. 2A and 2B schematically illustrate a method of manufacturing a microfluidic device using a photocurable resin, according to the present invention, and FIG. 2C depicts a photograph of a substrate and positions of microchannels;

FIG. 3 illustrates a procedure for an immunoassay;

FIG. 4 illustrates results of a measurement made using a disease marker, where FIG. 4A illustrates measurement results for fluorescence intensity, and FIG. 4B provides fluorescent micrographs where antigen concentration was changed and a reaction was carried out, using a device having CRP in the solid phase;

FIG. 5 illustrates results of a multiplex assay, where FIG. 5A illustrates results of a measurement where CEA concentration was changed, and FIG. 5B illustrates results of a measurement where CRP concentration was changed;

FIG. 6 illustrates another example, depicting an example using streptavidin, where FIG. 6A illustrates a control, FIG. 6B illustrates an exemplary test conducted with anti-EGFR antibody immobilized via streptavidin, and FIG. 6C illustrates an exemplary test conducted with anti-EGFR antibody immobilized directly;

FIG. 7 illustrates another example, depicting examples of immobilized specimens;

FIG. 8 schematically illustrates a conventional method of manufacturing a microfluidic device, where FIG. 8A illustrates a single-item analysis device, and FIG. 8B illustrates a triplex analysis device; and

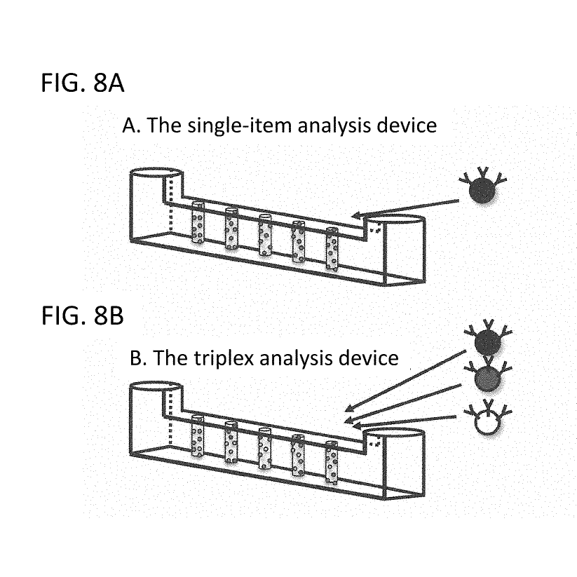

FIG. 9 schematically illustrates an experiment where a plurality of types of microbeads having antibodies are severally cured together with a photocurable resin, where FIGS. 9A to 9C are schematic drawings of an immobilized process of the plurality of types of microbeads, and FIG. 9D shows detection results.

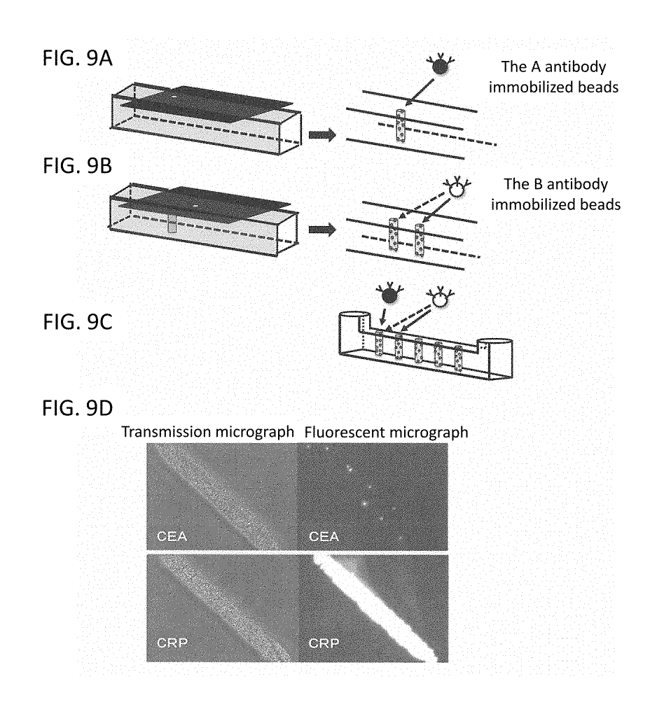

### DESCRIPTION OF THE PREFERRED EMBODIMENTS

In the following, the description of the present invention centers on a device for an immunoassay using an antibody. However, any specifically binding molecule may be used. For example, in the microfluidic device of the present invention (hereafter also referred to simply as “device”), any reagent that specifically binds to a substance targeted for detection may be used as a specific binding reagent and be immobilized. Examples may include an antibody, antigen, aptamer, DNA, RNA, or cell lysate. In the case of an antibody, the antibody molecule itself may be used or, as is the case with Fab and Fab₂, only a region specifically binding to an antigen may be used. In the case of an antigen, an antigen molecule may be used in its entirety, or may have a configuration that includes only an epitope region.

In order to bind the antibody to the device, a reagent specifically binding to the antibody (such as protein A or protein G) may serve as the specific binding reagent and may be immobilized. In addition, streptavidin may be immobilized to the device of the present invention, a biotinylated molecule such as a biotinylated antibody which binds to the substance to be detected may be immobilized in the device.

Any specimen that has the potential to contain a test substance may be used as a specimen that can be analyzed using the device of the present invention. For example, bodily fluids such as blood, blood serum, plasma, urine, or saliva; or extract solutions in which cells, tissue, or a scraped specimen have been extracted by a solvent such as physiological saline or a buffer solution can be used. When a sample contains blood cells or solid matter, such as with whole blood, saliva, or a tissue extract, a configuration may be employed that provides a prefilter at an inlet to a channel and is capable of filtration. In addition, in the case of fine cell debris or cell membranes, the sample can be used without filtration and binding can be confirmed.

Also, the immobilized specimen in the device can be used. Any specimen may be used as the immobilized specimen, but selecting a specimen capable of condensing is preferred from the vantage of sensitivity. Examples can include a cell, a cell cluster, a cell membrane, an organelle, an exosome, or the like. Because a specimen containing such materials is immobilized directly, it is extremely useful when, for example, investigating whether the substance sought for detection is actually present in the specimen. For example, when cells are mixed together with resin and cured, and the cells are sealed inside the resin, and a fluorescent-labeled antibody is introduced, it is possible to investigate what membrane protein is present on the cell. Accordingly, the device may serve as an extremely effective tool when used for research. Furthermore, information useful for the manufacture of the device can be obtained, such as to manufacture a device having a plurality of solid phase antibodies that recognize the same antigen and to select an antibody having favorable detection sensitivity, or to form a combination of antibodies for use in a sandwich assay, or the like.

Because the detection is highly sensitive, use of a fluorescent detector is preferred. In addition, detecting a single type of fluorescence suffices for a device system for microfluidics according to the present invention, and therefore a device for switching between fluorescences becomes unnecessary and the device can be made smaller. In the case of an apparatus provided with a placement platform on which the microfluidic device is placed, the apparatus measuring fluorescence intensity while scanning over the device and expressing the fluorescence intensity as a numerical value, no complex optical systems such as in a microscope are required, and therefore the apparatus can be reduced in price, and can be made smaller and lighter. A trial apparatus that was actually produced has a weight of approximately 1 kg, and is designed to be capable of driving even with a dry battery, and can be taken anywhere. In addition, by providing the fluorescent detector as a detection means, no intensive training is required such as with operating a microscope. For example, by initiating measurement via a measurement initiation means such as a start button and providing a display means on which data is displayed, a system can be built that enables anyone to take a measurement. Preferably, the fluorescent detector further includes a memory mechanism, and data is input for a calibration curve, in which a concentration of the specimen relative to fluorescence intensity was measured ahead of time. After fluorescent measurement, the concentration of the substance to be detected is immediately calculated, and displayed by the display means, whereby a user can learn the concentration of the substance to be detected in the specimen.

Any fluorescent label may be used, but a label having a wavelength that does not overlap with the intrinsic fluorescence of the substrate or resin is preferred. As an organic compound-type fluorescent label, a substance such as DyLight 650 (trademark) or the like having an excitation wavelength near 600 nm does not have a wavelength overlapping with the intrinsic fluorescence of the substrate, and therefore the background can be suppressed to a low level. An inorganic compound-type fluorescent label can also be used. The fluorescent lifespan of a quantum dot, for example, is extremely long and is therefore convenient for observation. In addition, a biomolecule-type fluorescent label, such as a protein, can also be used.

Any hydrophilic photocurable resin may be used as the photocurable resin used in the present invention. For example, a resin having an azide-type photosensitive group, a resin having at least two ethylenic unsaturated bonds in one molecule, or the like can be used. A water-soluble photocurable resin having at least two ethylenic unsaturated bonds in one molecule is preferably used that, generally, has a number-average molecular weight within a range of 300 to 30,000, and preferably within a range of 500 to 20,000; includes a sufficient ionic or non-ionic hydrophilic group that disperses uniformly in an aqueous medium (for example, a hydroxyl group, amino group, carboxy group, phosphate group, sulfonate group, ether linkage, or the like); and that, when irradiated with light having a wavelength in a range of approximately 250 to approximately 600 nm, cures and changes into a resin that is insoluble in water (see Patent Documents 2 to 5).

An example of a compound having an ethylenic unsaturated bond capable of photopolymerization at both ends of polyalkyl glycol can include the compounds noted below, for example, but are not limited to these.

Examples of representative hydrophilic photocurable resins include those noted below:

(1) Polyethylene glycol di(meth)acrylates having both-end hydroxyl groups of 1 mol polyethylene glycol with a molecular weight of 400 to 6000 esterified by 2 mol (meth)acrylate

(2) Polypropylene glycol di(meth)acrylates having both-end hydroxyl groups of 1 mol polypropylene glycol with a molecular weight of 200 to 4000 esterified by 2 mol (meth)acrylate

(3) Unsaturated polyethylene glycol urethanified product having both-end hydroxyl groups of 1 mol polyethylene glycol with a molecular weight of 400 to 6000 urethanified by 2 mol diisocyanate compound such as tolylene diisocyanate, xylylene diisocyanate, or isophorone diisocyanate, then 2 mol unsaturated monohydroxyethyl compound such as (meth)acrylate 2-hydroxyethyl or the like added

(4) Unsaturated polypropylene glycol urethanified product having both-end hydroxyl groups of 1 mol polypropylene glycol with a molecular weight of 200 to 4000 urethanified by 2 mol diisocyanate compound such as tolylene diisocyanate, xylylene diisocyanate, or isophorone diisocyanate, then 2 mol unsaturated monohydroxyethyl compound such as (meth)acrylate 2-hydroxyethyl or the like added.

As needed, a photopolymerization initiator is included in the hydrophilic photocurable resin. The photopolymerization initiator is a type of polymerization initiator and causes a cross-linking reaction between resins having a polymerizable unsaturated group. Examples can include α-carbonyls such as benzoin, acyloin ethers such as benzoin ethyl ether, polycyclic aromatic compounds such as naphthol, α-substituted acyloins such as methyl benzoin, and azoamide compounds such as 2-cyano-2-butyl azo formamide. In such a case, a proportion of the hydrophilic photocurable resin to the photopolymerization initiator used is not strictly limited, and can be altered across a broad range in accordance with the type of each component, and the like. In general, using a proportion of 0.1 to 5 parts by mass, and preferably 0.3 to 3 parts by mass, photopolymerization initiator to 100 parts by mass hydrophilic photocurable resin is appropriate.

In the present invention, an azide-unit pendant water-soluble photopolymer (AWP) is used as the photocurable resin. However, any resin capable of cross-linking with an amino group can be favorably used. In the following, a case using the AWP as the photocurable resin is described in detail. When the AWP is used, the AWP can be used at a volume ratio of 33 to 100% of the specific binding reagent or specimen. With a high resin concentration, a structure can be created that is unlikely to be washed away during washing, but the sensitivity of the device decreases. An optimum AWP concentration may be selected according to the affinity of the specific binding reagent for the target of the reagent. When an antibody is used as the specific binding reagent, the antibody is mixed with the resin at a concentration of 1 μg/mL to 10 mg/mL. The higher the antibody concentration, the higher the detection sensitivity the device can be created to have. The antibody concentration may be selected as appropriate according to the affinity of the antibody and/or detection sensitivity. In addition, the antibody and AWP are typically to be mixed at a volume ratio of 2:1, but a mixing ratio can also be selected as appropriate in accordance with the antibody to be used and/or sensitivity of the antibody to the substance to be detected.

The photocuring may use any type of ultraviolet ray irradiation apparatus with an irradiation intensity of approximately 20 mW/cm² near the 310 nm wavelength, and curing is performed for one second to three minutes. The amount of time for curing depends on the concentrations of the AWP, specific binding reagent, and specimen. The higher the AWP concentration, the shorter the amount of time for curing. In addition, curing of the resin can be confirmed using a phase contrast microscope or a differential interference microscope. A shape of the photocurable resin structure in which the specific binding reagent and the specimen have been mixed may be any shape, according to the shape of a photomask, such as a cylindrical pillar shape, a parallelepiped wall shape, or the like. In general, a shape having a larger specific surface area (ratio of the surface area of a reaction field to the volume of the specimen) has greater detection sensitivity and is therefore preferred.

Any type of substrate may be used, but because a photocurable resin is used, a substrate having high light transmittance is preferred. In addition, when a fluorescent label or the like is to be detected by optical measurement, a material having high transparency or a material that does not give off intrinsic fluorescence in the vicinity of the detection wavelength is appropriate. Of these, a cyclic olefin polymer substrate or a cyclic olefin copolymer substrate have a high degree of processing accuracy with injection molding, and are appropriate for the manufacture of microchannels by microfabrication. In addition, at least one microchannel is to be provided to the substrate.

As the washing solution that is used after the specific binding reagent is fixed, a blocking solution or a washing solution generally used in immunoassay can be used. For example, a phosphate buffer solution, Tris buffer solution, carbonate buffer solution, phosphate buffered saline (PBS), Tris buffered saline (TBS), or the like can be used as a buffer solution. In addition, when a protein such as an antibody is fixed to the device, in order to prevent nonspecific adsorption, a protein such as bovine serum albumen (BSA), skim milk, bovine serum, albumen, or the like can be used as a blocking agent. Also, when a nucleic acid such as DNA is fixed, a blocking agent such as salmon sperm DNA that is ordinarily used to block nucleic acid may be used as a blocking agent. Triton X-100, Tween 20, Brij-35, Nonidet P-40, SDS, or the like can be used as a surfactant.

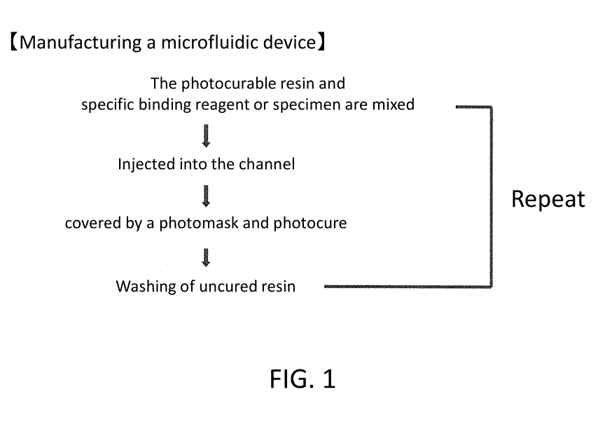

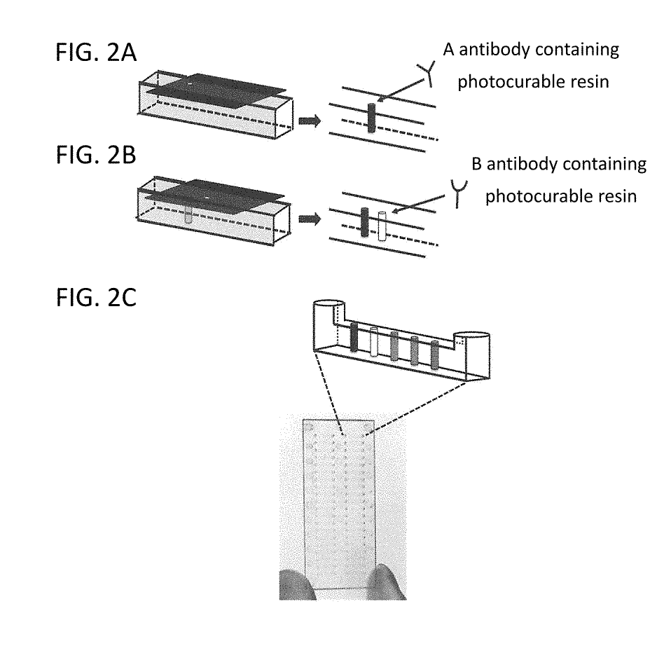

The microfluidic device is manufactured as illustrated in FIG. 1. A substrate is prepared having a channel formed thereon. The photocurable resin and specific binding reagent or specimen are mixed and filled in the channel of the device. The device is covered by a photomask worked with a design allowing light to pass only at a desired location, and the device is irradiated with ultraviolet rays to photocure the resin that is mixed with the specific binding reagent (FIG. 2A). Uncured resin is suctioned away and washing is performed with a washing solution. In a case where a multiplex specific binding reagent or the like is immobilized in the device, this procedure is repeated, and the specific binding reagent or the like, together with the photocurable resin, is fixed to the channel (FIG. 2B). After manufacturing the device, the channel may be filled with a washing solution and kept at a low temperature until assay. When a protein such as an antibody has been immobilized, if the device is kept humid at 4° C. without drying, the device is stable for approximately one year.

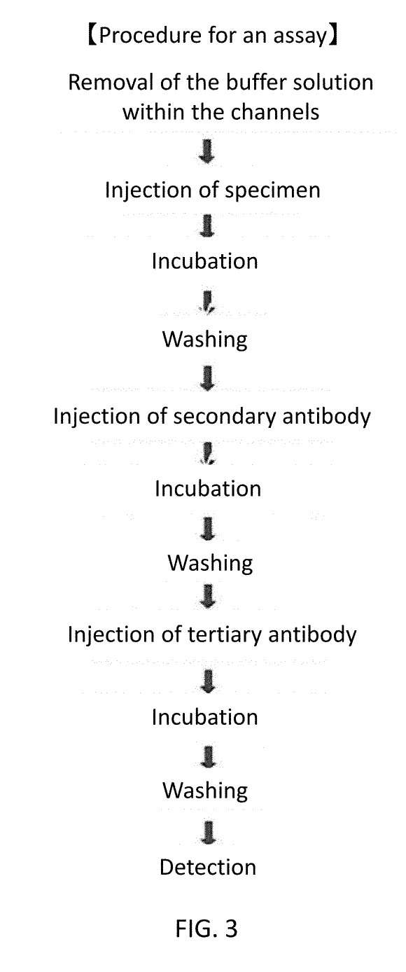

A representative assay method, the immunoassay, is now described. FIG. 3 depicts a procedure for a case where an antibody is fixed to the device and detection is performed. During manufacture, the channel of the device is filled with a buffer solution such as the washing solution or a blocking solution. Therefore, the buffer solution is removed. Next, the specimen is introduced to the channel and incubated. When the size of the channel is approximately 1000 μm×6500 μm×50 μm, 0.5 to 1.5 μL of the specimen is injected into one channel. Incubation at room temperature for approximately ten minutes is typically sufficient, although this is also dependent on the type of specimen and the concentration of the substance to be detected. Also, an incubator or the like that is set to 37° C. is used, whereby binding of the substance to be detected and the specific binding reagent can be performed in a shorter amount of time. Depending on the affinity of the specific binding reagent, which is fixated to the device, for the substance to be detected, and on the concentration of the substance to be detected, an amount of time for appropriate reaction may be set in a range of one minute to approximately 24 hours. Next, the specimen is suctioned away, and the washing solution is introduced and allowed to stand for approximately one minute. The washing solution is swapped out, and the same operation is repeated to perform washing. The washing operation is typically repeated approximately five times, whereby the device is completely cleaned. Also, the washing may be performed by swapping out the washing solution immediately, without allowing the solution to stand, and replacing the washing solution approximately seven to eight times. The number of washings and the amount of washing time may be adjusted as appropriate, depending on the substance to be detected and/or the specific binding reagent that is in the solid phase.

Next, a secondary antibody that binds to the substance to be detected is introduced to the channel. Although dependent on the concentration of the secondary antibody and the concentration of the substance to be detected, the reaction is completed in approximately 30 seconds. The secondary antibody is suctioned away, and the washing solution is introduced. The same washing operation described above is repeated. Next, a tertiary antibody labeled by a fluorescent label or the like is introduced to the channel and incubated. Typically, incubation for approximately 30 seconds is sufficient, similar to the secondary antibody. Thereafter, a similar washing is performed and the label is detected. When a tertiary antibody labeled by the fluorescent label is used, observation may be conducted with a fluorescent detector or fluorescent microscope. The amount of time required for the assay is 30 minutes or less. Accordingly, this is an extremely effective assay for a test or the like requiring urgency, where results must be produced in a short amount of time. In a case where the secondary antibody is given a fluorescent label, there is no need to use a tertiary antibody, and therefore assay time can be further reduced.

In the present invention, the phrase “labeled reagent binding specifically to the substance to be detected” may refer to the labeled secondary antibody binding to the substance to be detected, or to a combination that includes a labeled reagent capable of detecting the substance to be detected, as with a set of the secondary antibody binding specifically to the substance to be detected and the labeled tertiary antibody binding to the secondary antibody.

Here, a description was provided of an immunoassay having an antibody immobilized in a channel. However, in a case where the specimen is immobilized, detection may also be performed with a labeled antibody that binds specifically to the substance to be detected, and may also be performed through combination of the antibody that binds specifically to the substance to be detected and a labeled antibody that recognizes the specifically binding antibody. In addition, when a nucleic acid is immobilized, detection may be performed by hybridization, in accordance with the usual method.

The microfluidic device manufactured by the method of the present invention can immobilize a plurality of different specific binding reagents to respective microstructures without commingling, but the device can be used to fix a single specific binding reagent, as shall be apparent.

Hereafter, the present invention is described by way of examples, but the present invention is not limited to the examples, as shall be apparent.

### Example 1

<<Detection Limits of Various Antigens Using Microfluidic Device>>

A microchip substrate (70 mm×30 mm×1.25 mm) of a cyclic olefin polymer (BS-X2194, manufactured by Sumitomo Bakelite Co., Ltd.) was used (see photograph, FIG. 2C). Parallelepiped (1000 μm×6500 μm×50 μm) microchannels were provided to forty locations on the substrate. An inlet and an outlet of each microchannel had a diameter of 1.0 mm.

Microfluidic devices were manufactured for detecting each of prostate-specific antigen (PSA), a prostate cancer marker; C-reactive protein (CRP), an inflammation marker; and carcinoembryonic antigen (CEA), a tumor marker. Microfluidic devices singly fixed with an anti-PSA antibody, an anti-CRP antibody, or an anti-CEA antibody were manufactured, and the detection limit was investigated using a purified antigen.

The following reagents were used in the PSA assay.

Primary antibody: Anti-PSA antibody (manufactured by Abcam PLC, ab10189, 2 mg/mL)

Antigen: Human PSA (manufactured by Acris Antibodies, Inc., P117-7)

Secondary antibody: Anti-PSA antibody (manufactured by Cell Signaling Technology, Inc., 5365)

Tertiary antibody: DyLight 650 (trademark) labeled goat anti-rabbit IgG (manufactured by Abcam PLC, ab96902)

The following reagents were used in the CRP assay.

Primary antibody: Anti-CRP antibody (manufactured by Abcam PLC, ab136176, 2 mg/mL)

Antigen: CRP (manufactured by Acris Antibodies, Inc., P100-0)

Secondary antibody: Anti-CRP antibody (manufactured by Abcam PLC, ab31156)

Tertiary antibody: DyLight 650 (trademark) labeled goat anti-rabbit IgG (manufactured by Abcam PLC, ab96902)

The following reagents were used in the CEA assay.

Primary antibody: Anti-CEA antibody (manufactured by Abcam PLC, ab4451, 2 mg/mL)

Antigen: Human CEA (manufactured by R&D Systems, 4128-CM-050)

Secondary antibody: Anti-CEACAMS antibody (rabbit) (manufactured by Abcam PLC, ab131070)

Tertiary antibody: DyLight 650 (trademark) labeled goat anti-rabbit IgG (manufactured by Abcam PLC, ab96902)

All primary antibodies were mixed, at a concentration of 2 mg/mL, with the photocurable resin AWP such that the antibody had a volume ratio of 1:1 with the resin and were filled into the channels, and were irradiated with ultraviolet rays for five seconds and photocured by an ultraviolet ray irradiation apparatus.

Each of the antigens were diluted to a desired concentration by PBS containing 1% BSA, were filled into the channels, and were incubated at room temperature for ten minutes. Thereafter, washing was performed in seven to eight sessions using 10 μL of washing solution. Specifically, washing was performed as follows. The antigen was suctioned away with an aspirator. A micropipette drew in 10 μL of washing solution and filled the channels such that the channels were filled with approximately 1.3 μL of washing solution. Suctioning away the washing solution with the aspirator and filling the channels with the washing solution was repeated, and washing was performed using 10 μL of washing solution per channel. The washing solution used 0.5% bovine serum albumen (BSA) and 0.5% Tween 20-added PBS.

The secondary antibody and tertiary antibody were diluted to 50 μg/mL by PBS containing 1% BSA and were each filled into the channels for 30 seconds, after which washing was performed as described above with 10 μL of washing solution.

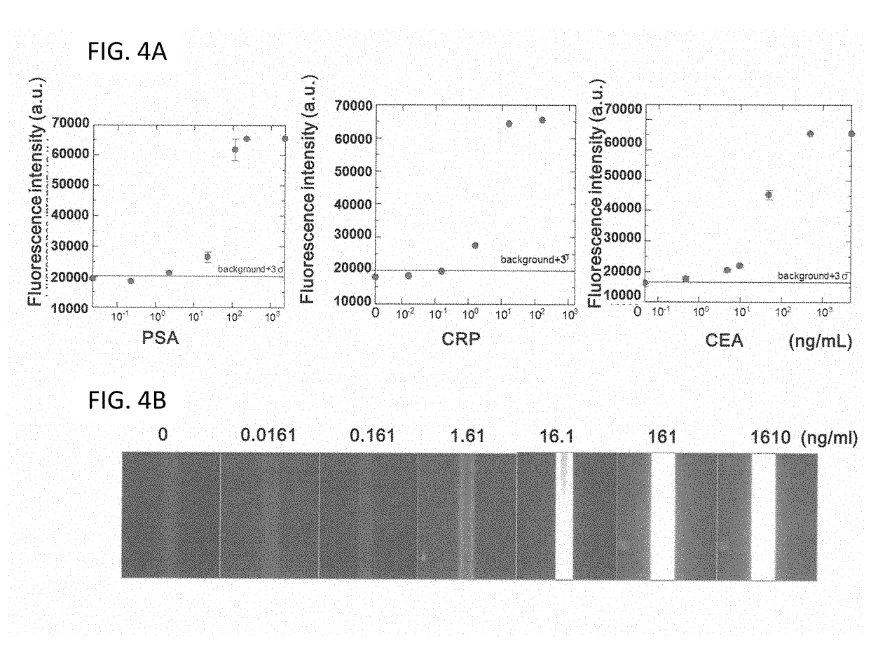

A fluorescent image was captured with a fluorescent microscope (manufactured by Nikon Corporation, Ni-E). As shown in FIG. 4A, clearly all of the antigens are detectable at extremely low concentration. The detection limits of PSA, CRP, and CEA calculated from these results were, respectively, 2.29 ng/mL, 1.61 ng/mL, and 0.49 ng/mL. A cut-off value of PSA for prostatic disease is 4 ng/mL. A cut-off value of CRP as an arteriosclerosis marker is 10 ng/mL. A cut-off value of CEA as a cancer marker is 5 ng/mL. Therefore, this device can clearly be applied to the diagnosis of diseases. Measuring each of the disease markers up to a detection limit sufficient for practical use was possible. In addition, the amount of time required from injection of the specimen through detection was 15 minutes, an extremely short amount of time.

FIG. 4B provides photographs of structures, using a CRP device similar to that described above, where the photocurable resin and antibody were fixed within the microchannels, antigen concentration was altered in a range of 0 to 1610 ng/mL and reaction was induced, after which the secondary antibody and tertiary antibody were reacted, observed using the fluorescent microscope. Using a high-pressure mercury lamp as a light source, and a Cy5 filter as a filter, imaging was performed with a digital CCD camera ORCA-R2 manufactured by Hamamatsu Photonics. The background was largely not observed, while fluorescence intensity was observed to increase dependent on specimen concentration. When CRP was 1.61 ng/mL or more, detection was indicated to be possible even with fluorescent microscope observation.

Although not given here, because a specific binding reagent such as an antibody is directly mixed with the photocurable resin and fixed in the device manufactured by the manufacturing method of the present invention, there is less variation among production lots of the device as compared to manufacture using microbeads.

### Example 2

<<Multiplex Detection Using Microfluidic Device>>

Next, a device that simultaneously detects a plurality of items was manufactured and investigated. A microfluidic device was manufactured having anti-CEA antibody and anti-CRP antibody placed in a single channel. The antibody used for immobilization, the antibody used in detection, and the antigen were the same as those used in Example 1.

With a procedure similar to that of Example 1, the anti-CEA antibody was mixed with the AWP resin, and the channels provided to the substrate were filled. Next, the device was covered by a photomask worked with a design allowing light to irradiate a desired location (hereafter referred to as a first region), the device was irradiated with ultraviolet rays to photocure the mixture of the resin and the antibody, and a wall-like parallelepiped structure was produced within the channels. The uncured resin was suctioned away and washing was performed with the washing solution.

Next, the anti-CRP antibody was mixed with the AWP resin and filled into the channels. The device was covered by a photomask worked with a design allowing light to irradiate a different region from the first region where the anti-CEA antibody was fixed (hereafter referred to as a second region), and the device was irradiated with ultraviolet rays to photocure the mixture of the resin and the anti-CRP antibody. A device for assaying a combination of items was manufactured having, in the same channels, the anti-CEA antibody fixed in the first region and the anti-CRP antibody fixed in the second region.

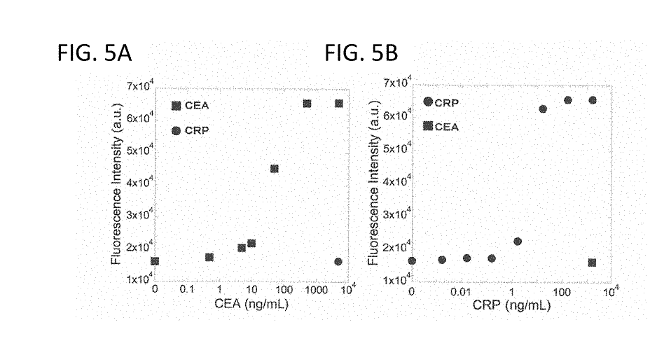

Each of the antigens and antibodies used were the same as those used in Example 1. Each of the antigens were filled in the channels at various concentrations and reacted, and the secondary antibody was mixed in and reacted such that the anti-CRP antibody and anti-CEA antibody each achieved a concentration of 50 μg/mL. Because the secondary antibody was a rabbit antibody, detection was performed with DyLight 650 (trademark) labeled goat anti-rabbit IgG as the tertiary antibody. A fluorescent microscope (manufactured by Nikon Corporation, Ni-E) was used for detection. Results are shown in FIG. 5.

FIG. 5 illustrates results of measurements made with the multiplex assay device manufactured in the foregoing, where the concentrations of CEA antigen and CRP antigen were changed. A measurement result for a region of the first region where the anti-CEA antibody was fixated is indicated by ▪, and a measurement result for a region of the second region where the anti-CRP antibody was fixated is indicated by ●.

FIG. 5A illustrates the fluorescence intensity measured when the concentration of the CEA antigen was changed, and FIG. 5B illustrates fluorescence intensity measured when the concentration of the CRP antigen was changed. As depicted in FIG. 5A, with the CEA antigen, an increase in fluorescence intensity was observed dependent on the concentration in the first region (▪), where the anti-CEA antibody was in the solid phase. Meanwhile, even when an elevated CEA antigen concentration of 5000 ng/mL was used, a fluorescence intensity equivalent to the background level was all that was observed in the second region (●), where the anti-CRP antibody is in the solid phase.

As depicted in FIG. 5B, when the CRP antigen concentration was changed and the fluorescence intensity was measured, all that was measured in the first region (▪), where the anti-CRP antibody was not in the solid phase, was a background level fluorescence intensity. In other words, this indicates that only the anti-CEA antibody was in the solid phase in the first region and only the anti-CRP antibody was in the solid phase in the second region. Also, as depicted in FIG. 5, both of the antigens were detectable up to an extremely low-concentration detection limit, similar to the single-item assay device depicted in FIG. 4.

As illustrated in Example 2, assays of the same specimen can be performed simultaneously, and therefore the assay is appropriate for measurement of items where simultaneous assay is preferred, as with the anti-CRP antibody and the anti-CEA antibody. In this example, a composite assay of two types of antibody was illustrated. However, by immobilizing the antibodies to be detected simultaneously, any number of antibodies can be incorporated into the microfluidic device.

### Example 3

An example is given of a specific binding reagent other than an antibody immobilizing in the channels. Streptavidin was used as the specific binding reagent to be immobilized to the microchannels. Streptavidin was dissolved in PBS (pH 7.4) to a concentration of 10 mg/mL, and was mixed at a volume ratio of 1 to 1 with AWP. After mixing, the mixture was cured by ultraviolet rays as in Example 1.

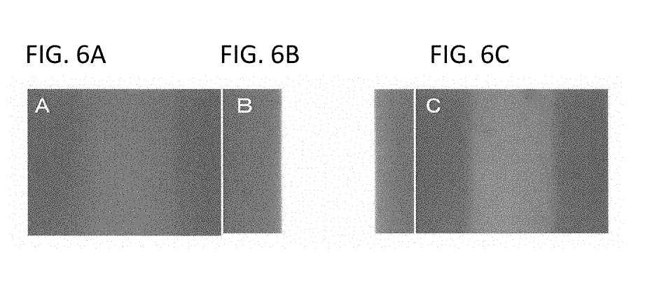

Next, the primary antibody (biotin-modified anti-EGFR antibody, manufactured by Abcam PLC, ab113645) having a concentration of 50 μg/mL was filled into the channels, and a streptavidin-biotin binding reaction was performed for one hour at room temperature. Thereafter, the anti-EGFR antibody was suctioned away with an aspirator and washing was performed with 10 μL of washing solution, after which the device was used in the assay. A specimen having pleural fluid sediment from a lung cancer patient dissolved in Lysis Buffer (manufactured by Cell Signaling Technology, Inc., 9803) was used for the antigen-containing specimen. Each of the secondary antibody (an anti-L858R gene mutant EGFR antibody (manufactured by Cell Signaling Technology, Inc., 3197)) and the tertiary antibody (DyLight 650 (trademark) labeled rabbit anti-goat IgG (manufactured by Abcam PLC, ab102343)) were diluted with PBS containing 1% BSA and adjusted to 50 μg/mL before use. The assay was conducted with a procedure similar to that of Example 1, and results are given in FIG. 6. FIGS. 6A and 6B are fluorescent micrographs of regions where the anti-EGFR antibody is immobilized via streptavidin, and FIG. 6C is a fluorescent micrograph of a region where the anti-EGFR antibody is immobilized directly. FIG. 6A depicts, as a control, a region where no specimen was used when carrying out the subsequent assay. FIGS. 6B and 6C depict assays that were carried out identically from the point of incubation with the specimen.

As illustrated in FIG. 6, by using streptavidin, a sensitivity was obtained that was approximately one hundred times higher than when the anti-EGFR antibody is immobilized directly. It is believed that this is because, due to the ability to use streptavidin, which has a low molecular weight and high concentration, the antibodies can be fixed at a high density.

As has been illustrated above, any substance, not just an antibody, that exhibits specific binding may be used and fixed to the solid phase. Also, because no contamination occurs between regions, favorably sensitive detection of a target is possible.

### Example 4

Next, an example is given of the immobilized specimen. Detection was performed for EML4-ALK fusion protein, which is a cause in lung cancer. The EML4-ALK fusion protein is an abnormal protein in which EML4 gene and ALK gene are fused, and approximately half of the amino terminals of the EML4 protein are fused with an intracellular region of ALK receptor tyrosine kinase. Cell line H3122, which expresses EML4-ALK fusion protein, and cell line H358, which does not express EML4-ALK fusion protein, were used. The collected culture cells were separated into cells and supernatant by centrifugal separation, the supernatant was removed, and Lysis Buffer (manufactured by Cell Signaling Technology, Inc., 9803) was added to the cells, whereby the cell lysate was prepared.

AWP and the cell lysates prepared from the two cell lines were mixed at a 1:1 volume ratio, each mixture was filled into the microchannels, and was exposed to ultraviolet rays and photocured. Washing was performed with a washing solution, and a device was manufactured having microstructures with each of the cell lysates in the solid phase.

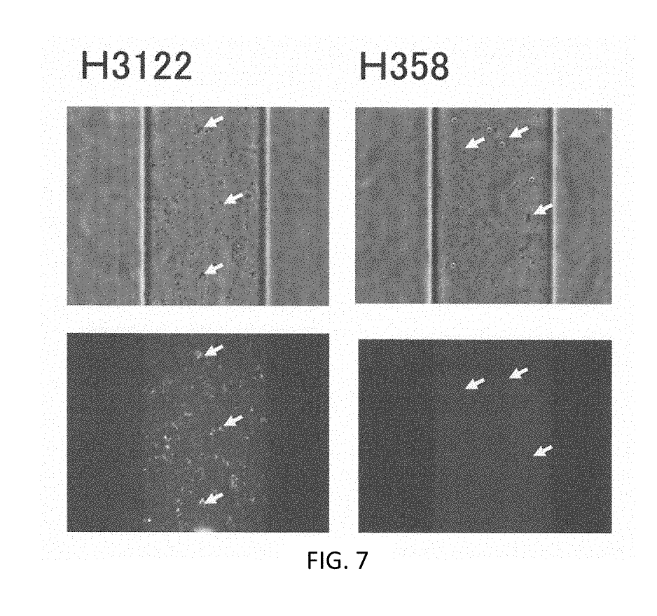

Detection was performed as follows. A mouse antibody (manufactured by Santa Cruz, SC-57024) that specifically binds to the EML4-ALK fusion protein was introduced to the channels, and was incubated for 30 seconds. Washing was performed with the washing solution, after which DyLight 650 labeled anti-mouse IgG antibody (manufactured by Abcam PLC, ab98797) was introduced to the channels, and was incubated for 30 seconds. Washing was performed with the washing solution, and observation was conducted with a fluorescent microscope and bright-field. Results are shown in FIG. 7.

As indicated by the bright-field images at the top of FIG. 7, fragments of cell membrane were observed in each of the devices using the H3122 and H358 cell lysates in the solid phase (portions of the observed cell membranes are indicated in the drawing by arrows). However, in the fluorescent micrographs at the bottom of FIG. 7, fluorescence was observed only with H3122, which are the EML4-ALK fusion protein-positive cells. The observed fluorescence matches the region where black spots indicating the cell membranes are observed in the bright-field image (corresponding positions in the fluorescence micrographs are indicated by arrows).

As noted above, due to the specimen is immobilized directly, the inclusion of the substance to be detected can be confirmed. In addition, because the specific binding of the antibody to the specimen can be confirmed, the device is useful when selecting a highly specific antibody or highly binding antibody for use in an assay.

### INDUSTRIAL APPLICABILITY

According to the method of the present invention, detection with favorable sensitivity is possible in a short amount of time, and a detector can be made smaller even when testing a plurality of items. Accordingly, the device is extremely useful in point-of-care testing (POCT) where testing is performed at a patient's bedside, and when test results are required in a short amount of time. In addition, due to the specimen is immobilized directly, the inclusion of the substance to be detected can be confirmed, and the specific binding of the antibody to the substance to be detected can be confirmed in an extremely short amount of time.

1-9. (canceled)

10. A microfluidic device having at least one channel on a substrate;
a plurality of microstructures being arranged within each of the channels with a gap provided between the microstructures and a channel side wall, and, in the microstructures, one type of specific binding reagent comprising an amino group or one type of specimen comprising an amino group mixed into a photocured hydrophilic resin having an azide-type photosensitive group being directly held in the hydrophilic resin through cross-linking.

11. The microfluidic device of claim 10, wherein, in each of the microstructures, a different specific binding reagent and/or specimen is directly held in the hydrophilic resin through cross-linking.

12. The microfluidic device of claim 10, wherein the specific binding reagent is at least one of an antibody, antigen, avidin, streptavidin, or biotin.

13. The microfluidic device of any one of claim 10, wherein the specimen comprises a cell, cell cluster, cell membrane, organelle, or exosome.

14. An analysis kit, comprising:
the microfluidic device of any claim 10; and
a labeled reagent that binds specifically to a substance to be detected.

15. The analysis kit of claim 14, wherein the labeled reagent can be detected by a single optical system.

16. A method of manufacturing a microfluidic device, comprising:
a substrate preparation step of preparing a substrate comprising at least one channel;
a filling step of filling an interior of the channel with a mixed solution of one type of specific binding reagent comprising an amino group or one type of specimen comprising an amino group and a hydrophilic photocurable resin having an azide-type photosensitive group;
an exposure step of performing exposure to light using a photomask in which a structure is formed in a site which is a portion of the hydrophilic photocurable resin filled into the channel with a gap being provided between the site and a channel side wall, and of cross-linking and photocuring the specific binding reagent or specimen directly with a resin;
a washing step of removing and washing uncured resin from the channel;
a refilling step of filling the interior of the channel with a mixed solution of a specific binding reagent or specimen different from the said specific binding reagent or specimen and a hydrophilic photocurable resin;
a re-exposure step of exposing the hydrophilic photocurable resin filled into the channel to light using a photomask in which a structure is formed on a site where uncured photocurable resin is present, with a gap being provided between the site and the channel side wall;
a re-washing step of removing and washing uncured resin from the channel; and
repeating the steps from the refilling step to the re-washing step and fixing a plurality of different specific binding reagents and/or specimens inside the channel.

17. A microfluidic device having at least one channel on a substrate,
at least one microstructure being arranged within each of the channels with a gap provided between the microstructure and a channel side wall, and, in the microstructures, one type of specific binding reagent comprising an amino group or one type of specimen comprising an amino group mixed into a photocured hydrophilic resin having an azide-type photosensitive group being held directly in the hydrophilic resin through cross-linking.

18. The microfluidic device of claim 10, wherein the hydrophilic resin is an azide-unit pendant water-soluble photopolymer.

19. A detection method using the microfluidic device of claim 10, the method comprising:
an incubation step of filling the channel of the microfluidic device with a specimen or specific binding reagent;
a washing step of washing the specimen or specific binding reagent;
a labeling step of performing labeling with a fluorescent-labeled labeling reagent that specifically binds to the substance to be detected;
a washing step of washing the labeling reagent; and
a step of performing detection using a detection means comprising a means for detecting only one fluorescence.

20. The microfluidic device of claim 17, wherein the specific binding reagent is at least one of an antibody, antigen, avidin, streptavidin, or biotin.

21. The microfluidic device of claim 17, wherein the specimen comprises a cell, cell cluster, cell membrane, organelle, or exosome.

22. An analysis kit, comprising:
the microfluidic device of claim 17; and
a labeled reagent that binds specifically to a substance to be detected.

23. The analysis kit of claim 22, wherein the labeled reagent can be detected by a single optical system.

24. The microfluidic device of claim 17, wherein the hydrophilic resin is an azide-unit pendant water-soluble photopolymer.

25. A detection method using the microfluidic device of claim 17, the method comprising:
an incubation step of filling the channel of the microfluidic device with a specimen or specific binding reagent;
a washing step of washing the specimen or specific binding reagent;
a labeling step of performing labeling with a fluorescent-labeled labeling reagent that specifically binds to the substance to be detected;
a washing step of washing the labeling reagent; and
a step of performing detection using a detection means comprising a means for detecting only one fluorescence.
